(19) United States
(12) Patent Application Publication
(10) Pub. No.: US 2007/0270446 A1
(43) Pub. Date: Nov. 22, 2007

(54) Reversed Pyrimidinone Compounds as Calcilytics

(75) Inventors: Robert W. Marquis Jr, Collegeville, PA (US); Jae U. Jeong, Collegeville, PA (US); Juan I Luengo, Collegeville, PA (US); Dennis Shinji Yamashita, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION, Philadelphia, PA (US)

(21) Appl. No.: 11/663,238

(22) PCT Filed: Oct. 6, 2005
(86) PCT No.: PCT/US05/35906

§ 371 Date: Jul. 27, 2007

Related U.S. Application Data
(60) Provisional application No. 60/616,389, filed on Oct. 6, 2004.

(51) Int. Cl. A61K 31/505; A61P 19/08; C07D 239/02

(52) U.S. Cl. 514/269; 544/319

(57) ABSTRACT

Calcilytic compounds and methods of preparing them are disclosed. Methods of using the calcilytic compounds are also provided.

### TECHNICAL FIELD

The present invention relates to reversed pyrimidinone compounds able to inhibit calcium receptor activity, pharmaceutical compositions containing these compounds, and methods for preparing the compounds and compositions. The present invention also relates to the uses of such compounds and compositions, particularly their use in administering to patients to achieve a therapeutic effect.

### BACKGROUND OF THE INVENTION

In mammals, extracellular Ca²⁺ is under rigid homeostatic control and regulates various processes such as blood clotting, nerve and muscle excitability, and proper bone formation. Extracellular Ca²⁺ inhibits the secretion of parathyroid hormone (“PTH”) from parathyroid cells, inhibits bone resorption by osteoclasts, and stimulates secretion of calcitonin from C-cells. Calcium receptor proteins enable certain specialized cells to respond to changes in extracellular Ca²⁺ concentration.

PTH is the principal endocrine factor regulating Ca²⁺ homeostasis in the blood and extracellular fluids. PTH, by acting on bone and kidney cells, increases the level of Ca²⁺ in the blood. This increase in extracellular Ca²⁺ then acts as a negative feedback signal, depressing PTH secretion. The reciprocal relationship between extracellular Ca²⁺ and PTH secretion forms an important mechanism maintaining bodily Ca²⁺ homeostasis.

Extracellular Ca²⁺ acts directly on parathyroid cells to regulate PTH secretion. The existence of a parathyroid cell surface protein which detects changes in extracellular Ca²⁺ has been confirmed. See Brown et al., Nature 366:574, 1993. In parathyroid cells, this protein, the calcium receptor, acts as a receptor for extracellular Ca²⁺, detects changes in the ion concentration of extracellular Ca²⁺, and initiates a functional cellular response, PTH secretion.

Extracellular Ca²⁺ influences various cell functions, reviewed in Nemeth et al., Cell Calcium 11:319, 1990. For example, extracellular Ca²⁺ plays a role in parafollicular (C-cells) and parathyroid cells. See Nemeth, Cell Calcium 11:323, 1990. The role of extracellular Ca²⁺ on bone osteoclasts has also been studied. See Zaidi, Bioscience Reports 10:493, 1990.

Various compounds are known to mimic the effects of extra-cellular Ca²⁺ on a calcium receptor molecule. Calcilytics are compounds able to inhibit calcium receptor activity, thereby causing a decrease in one or more calcium receptor activities evoked by extracellular Ca²⁺. Calcilytics are useful as lead molecules in the discovery, development, design, modification and/or construction of useful calcium modulators, which are active at Ca²⁺ receptors. Such calcilytics are useful in the treatment of various disease states characterized by abnormal levels of one or more components, e.g., polypeptides such as hormones, enzymes or growth factors, the expression and/or secretion of which is regulated or affected by activity at one or more Ca²⁺ receptors. Target diseases or disorders for calcilytic compounds include diseases involving abnormal bone and mineral homeostasis.

Abnormal calcium homeostasis is characterized by one or more of the following activities: an abnormal increase or decrease in serum calcium; an abnormal increase or decrease in urinary excretion of calcium; an abnormal increase or decrease in bone calcium levels (for example, as assessed by bone mineral density measurements); an abnormal absorption of dietary calcium; an abnormal increase or decrease in the production and/or release of messengers which affect serum calcium levels such as PTH and calcitonin; and an abnormal change in the response elicited by messengers which affect serum calcium levels.

Thus, calcium receptor antagonists offer a unique approach towards the pharmacotherapy of diseases associated with abnormal bone or mineral homeostasis, such as hypoparathyroidism, osteosarcoma, periodontal disease, fracture healing, osteoarthritis, joint replacement, rheumatoid arthritis, Paget's disease, humoral hypercalcemia associated with malignancy and fracture healing, and osteoporosis.

### SUMMARY OF THE INVENTION

Reversed pyrimidinone compounds are disclosed herein which are useful as calcium receptor antagonists in the treatment of a variety of diseases associated with abnormal bone or mineral homeostasis, including but not limited to hypoparathyroidism, osteosarcoma, periodontal disease, fracture healing, osteoarthritis, joint replacement, rheumatoid arthritis, Paget's disease, humoral hypercalcemia associated with malignancy and fracture healing, and osteoporosis. The compounds are represented by Formula (I) hereinbelow

A method for antagonizing calcium receptors in an animal, including humans, is also disclosed. The method comprises administering to an animal in need thereof an effective amount of a compound of Formula (I), indicated hereinbelow.

A method for increasing serum parathyroid levels in an animal, including humans, is additionally disclosed. The method comprises administering to an animal in need thereof an effective amount of a compound of Formula (I), indicated herein below.

### DETAILED DESCRIPTION OF PREFERRED EMBODIMENTS

Reverse pyrimidinone compounds are disclosed herein which are useful as calcilytic compounds or calcilytics. “Calcilytics” and “calcilytic compounds” refer to compounds able to inhibit calcium receptor activity. The ability of a compound to “inhibit calcium receptor activity” means that the compound causes a decrease in one or more calcium receptor activities evoked by extracellular Ca²⁺.

The use of calcilytic compounds to inhibit calcium receptor activity and/or achieve a beneficial effect in a patient are described below. More specifically, the present application demonstrates the ability of calcilytic compounds to increase PTH secretion, thereby confirming that the parathyroid gland calcium receptor is a target site for these compounds. Also described below are techniques which can be used to obtain additional calcilytic compounds.

Examples of the featured calcilytic compounds are provided by the chemical formula depicted in Structure I and the accompanying description.

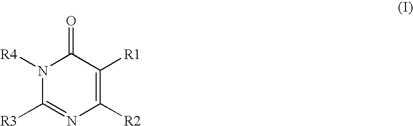

wherein:

R⁴ and R³ are independently one of: H, halogen, CN, CF₃, lower alkyl, cycloalky, or aryl; or R⁴ and R³ are together —(CH₂)ₙ— and n is 5, 4, or 3;

R² is an aryl group, which may have 0 to 4 substituents in the aryl ring and each substituent is at least one of: halogen, CN, CF₃, OCF₃, lower alkyl, N(lower alkyl)₂, lower alkoxy, OH, OC(O)-lower alkyl, OC(O)-lower alkylamino, or OC(O)-lower alkyl-N(lower alkyl)₂;

R¹ is at least one of lower alkyl, aryl or a group of the formula —(CH₂)ₙ—R⁵ wherein n is 0, 1, or 2; R⁵ is an aryl group which may have 0 to 3 substituents on the aryl ring and each substituent is at least one of: halogen, CN, CF₃, OCF₃, lower alkyl, lower alkoxy, NH-lower alkyl, NH-alkylaryl, N(lower alkyl)₂, OH, OC(O)-lower alk, OC(O)-lower alkylamino, or OC(O)-lower alkyl-N(lower alk)₂; or

pharmaceutically acceptable salts, hydrates, tautomers, solvates or complexes thereof.

As used herein, “alkyl” refers to an optionally substituted hydrocarbon group joined by single carbon-carbon bonds and having 1-20 carbon atoms joined together. The alkyl hydrocarbon group may be linear, branched or cyclic, saturated or unsaturated. Substituents on optionally substituted alkyl may be at least one of: aryl, CO₂R, CO₂NHR, OH, OR, CO, NH₂, halo, CF₃, OCF₃ or NO₂, wherein R represents H, C₁₋₄ alkyl, C₃₋₆ cycloalkyl, C₂₋₅ alkenyl, C₂₋₅ alkynyl, heterocycloalkyl, or aryl. Additional substituents may be at least one of: F, Cl, Br, I, N, S or O. In one embodiment, no more than three substituents are present. In another embodiment, the alkyl has 1-12 carbon atoms and is unsubstituted. The alkyl group may be linear.

As used herein “cycloalkyl” refers to optionally substituted 3-7 membered carbocyclic rings wherein any substituents may be at least one of, F, Cl, Br, I, N(R₄)₂, SR₄ or OR₄, unless otherwise indicated.

As used herein, “aryl” refers to an optionally substituted aromatic group with at least one ring having a conjugated pi-electron system, containing up to two conjugated or fused ring systems. Aryl includes carbocyclic aryl, and biaryl groups, all of which may be optionally substituted. Phenyl and naphthyl are particularly useful aryl, especially phenyl. Examples of suitable substituents include at least one of: halogen, C₁₋₄ alkyl, OCF₃, CF₃, OMe, CN, OSO₂R or NO₂, wherein R represents C₁₋₄ alkyl or C₃₋₆ cycloalkyl.

As used herein, “heteroaryl” refers to an aryl ring containing 1, 2 or 3 heteroatoms such as N, S, or O.

As used herein, “alkenyl” refers to an optionally substituted hydrocarbon group containing at least one carbon-carbon double bond and containing up to 5 carbon atoms joined together. The alkenyl hydrocarbon chain may be straight, branched or cyclic. Any substituents are at least one of halogen, C₁₋₄ alkyl, OCF₃, CF₃, OMe, CN, OSO₂R or NO₂, wherein R represents C₁₋₄ alkyl or C₃₋₆ cycloalkyl.

As used herein, “alkynyl” refers to an optionally substituted hydrocarbon group containing at least one carbon-carbon triple bond between the carbon atoms and containing up to 5 carbon atoms joined together. The alkynyl hydrocarbon group may be straight-chained, branched or cyclic. The substituents are at least one of: halogen, C₁₋₄ alkyl, OCF₃, CF₃, OMe, CN, OSO₂R or NO₂, wherein R represents C₁₋₄ alkyl or C₃₋₆ cycloalkyl.

The reversed pyrimidinone compound may contain one or more asymmetric carbon atoms and may exist in racemic and optically active forms. All of these compounds and diastereomers are contemplated to be within the scope of the present invention.

Examples of reversed pyrimidinone compounds include:
6-(2-hydroxyphenyl)-2-methyl-5-(2-phenylethyl)-3-propyl-4(3H)-pyrimidinone;
6-(2-hydroxyphenyl)-2-methyl-5-(2-phenylethyl)-3-ethyl-4(3H)-pyrimidinone;
6-(2-hydroxyphenyl)-2-methyl-5-(2-phenylethyl)-3-methyl-4(3H)-pyrimidinone;
6-(2-hydroxyphenyl)-2-methyl-5-(2-phenylethyl)-3-[2-(2-pyridinyl)ethyl]-4(3H)-pyrimidinone;
6-(2-hydroxyphenyl)-2-methyl-5-(2-phenylethyl)-3-butyl-4(3H)-pyrimidinone;
6-(2-hydroxyphenyl)-2-methyl-5-(2-phenylethyl)-3-pentyl-4(3H)-pyrimidinone;
6-(2-hydroxy-phenyl)-2-methyl-5-(2-phenethyl)-3-hexyl-3H-pyrimidin-4-one;
3-cyclopropylmethyl-6-(2-hydroxy-phenyl)-2-methyl-5-phenethyl-3H-pyrimidin-4-one;
3-(2-methylallyl)-6-(2-hydroxy-phenyl)-2-methyl-5-phenethyl-3H-pyrimidin-4-one;
3-(3-methylbutyl)-6-(2-hydroxy-phenyl)-2-methyl-5-phenethyl-3H-pyrimidin-4-one;
3-(2-cyclohexylethyl)-6-(2-hydroxy-phenyl)-2-methyl-5-phenethyl-3H-pyrimidin-4-one;
3-propyl-6-(3-fluoro-2-hydroxy-phenyl)-2-methyl-5-phenethyl-3H-pyrimidin-4-one;
3-hexyl-6-(3-fluoro-2-hydroxy-phenyl)-2-methyl-5-phenethyl-3H-pyrimidin-4-one;
3-propyl-6-(2-hydroxy-phenyl)-2-methyl-5-(2-cyclohexylethyl)-3H-pyrimidin-4-one;
2-(2-hydroxyphenyl)-3-(2-phenylethyl)-6,7,8,9-tetrahydro-4H-pyrido[1,2-a]pyrimidin-4-one;
3-(2-cyclo hexylethyl)-2-(2-hydroxyphenyl)-6,7,8,9-tetra hydro-4H-pyrido[1,2-a]pyrimidin-4-one;
3-cyclopropyl-6-(2-hydroxyphenyl)-2-methyl-5-(2-phenylethyl)pyrimidin-4(3H)-one;
6-(2-hydroxyphenyl)-2-methyl-3-[2-(1-methylpyrrolidin-2-yl)ethyl]-5-(2-phenylethyl)pyrimidin-4(3H)-one;
3-(2,2-dimethylpropyl)-6-(2-hydroxyphenyl)-2-methyl-5-(2-phenylethyl)pyrimidin-4(3H)-one;
3-sec-butyl-6-(2-hydroxyphenyl)-2-methyl-5-(2-phenylethyl)pyrimidin-4(3H)-one;
3-cyclopentyl-6-(2-hydroxyphenyl)-2-methyl-5-(2-phenylethyl)pyrimidin-4(3H)-one;
6-(2-hydroxyphenyl)-3-isobutyl-2-methyl-5-(2-phenylethyl)pyrimidin-4(3H)-one;
3-cyclobutyl-6-(2-hydroxyphenyl)-2-methyl-5-(2-phenylethyl)pyrimidin-4(3H)-one;
3-cyclohexyl-6-(2-hydroxyphenyl)-2-methyl-5-(2-phenylethyl)pyrimidin-4(3H)-one;
6-(2-hydroxyphenyl)-3-isopropyl-2-methyl-5-(2-phenylethyl)pyrimidin-4(3H)-one;
6-(2-hydroxyphenyl)-2-methyl-5-(2-phenylethyl)-3-(2,2,2-trifluoroethyl)pyrimidin-4(3H)-one;
6-(2-hydroxyphenyl)-2-methyl-3-octyl-5-(2-phenylethyl)pyrimidin-4(3H)-one;
3-heptyl-6-(2-hydroxyphenyl)-2-methyl-5-(2-phenylethyl)pyrimidin-4(3H)-one;
3-allyl-6-(2-hydroxyphenyl)-2-methyl-5-(2-phenylethyl)pyrimidin-4(3H)-one;
6-(3-fluoro-2-hydroxyphenyl)-2,3-dimethyl-5-(2-phenylethyl)pyrimidin-4(3H)-one;
3-ethyl-6-(3-fluoro-2-hydroxyphenyl)-2-methyl-5-(2-phenylethyl)pyrimidin-4(3H)-one;
3-butyl-6-(3-fluoro-2-hydroxyphenyl)-2-methyl-5-(2-phenylethyl)pyrimidin-4(3H)-one;
2-(dimethylamino)-6-(2-hydroxyphenyl)-5-(2-phenylethyl)-4(1H)-pyrimidinone;
6-(2-hydroxyphenyl)-2-methyl-3-phenyl-5-(2-phenylethyl)-4(3H)-pyrimidinone;
6-(3-fluoro-2-hydroxyphenyl)-3-heptyl-2-methyl-5-(2-phenylethyl)-4(3H)-pyrimidinone;
3-(1-benzothien-2-yl)-6-(3-fluoro-2-hydroxyphenyl)-2-methyl-5-(2-phenylethyl)-4(3H)-pyrimidinone;
6-(3-fluoro-2-hydroxyphenyl)-2-methyl-3-(5-methyl-2-thienyl)-5-(2-phenylethyl)-4(3H)-pyrimidinone;
6-(3-fluoro-2-hydroxyphenyl)-2-methyl-3-(4-methyl-2-thienyl)-5-(2-phenylethyl)-4(3H)-pyrimidinone;
3-(4-biphenylyl)-6-(3-fluoro-2-hydroxyphenyl)-2-methyl-5-(2-phenylethyl)-4(3H)-pyrimidinone; and
6-(3-fluoro-2-hydroxyphenyl)-2-methyl-5-(2-phenylethyl)-3-(5-phenyl-2-thienyl)-4(3H)-pyrimidinone.

Pharmaceutically acceptable salts are non-toxic salts in the amounts and concentrations at which they are administered.

Pharmaceutically acceptable salts include acid addition salts such as those containing sulfate, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Hydrochloride is a particularly useful pharmaceutically acceptable salt. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethylenediamine, meglumine, procaine, aluminum, calcium, lithium, magnesium, potassium, sodium, ammonium, alkylamine, and zinc, when acidic functional groups, such as carboxylic acid or phenol are present.

The compounds of Formula (I) above can be prepared using standard techniques. An overall strategy for preparing preferred compounds described herein can be carried out as described in this section. The examples, which follow, illustrate the synthesis of specific compounds. Using the protocols described herein as a model, one of ordinary skill in the art can readily produce other compounds of the present invention.

All reagents and solvents were obtained from commercial vendors. Starting materials were synthesized using standard techniques and procedures.

Synthesis Schemes

The synthesis of pyrimidinones covered in this application may be achieved by one of the two methods adumbrated below in Schemes 1 or 2. The β-keto ester 3 may be synthesized by methods common in the art. Treatment of ester 1 with sodium hydride followed by addition of the aromatic ester 2 provides the β-keto-ester 3. Treatment of 3 with acetamidine in the presence of a base such as sodium methoxide or potassium carbonate provides pyrmidinone 4. Treatment of 4 with a base such as sodium hydride in the presence of lithium bromide and an alkylating agent such as 1-bromopropane provides 5. De-protection of the methyl ether protecting group of 5 may be achieved by methods common to the art such as treatment with boron tribromide to provide the target pyrimidinone 6.

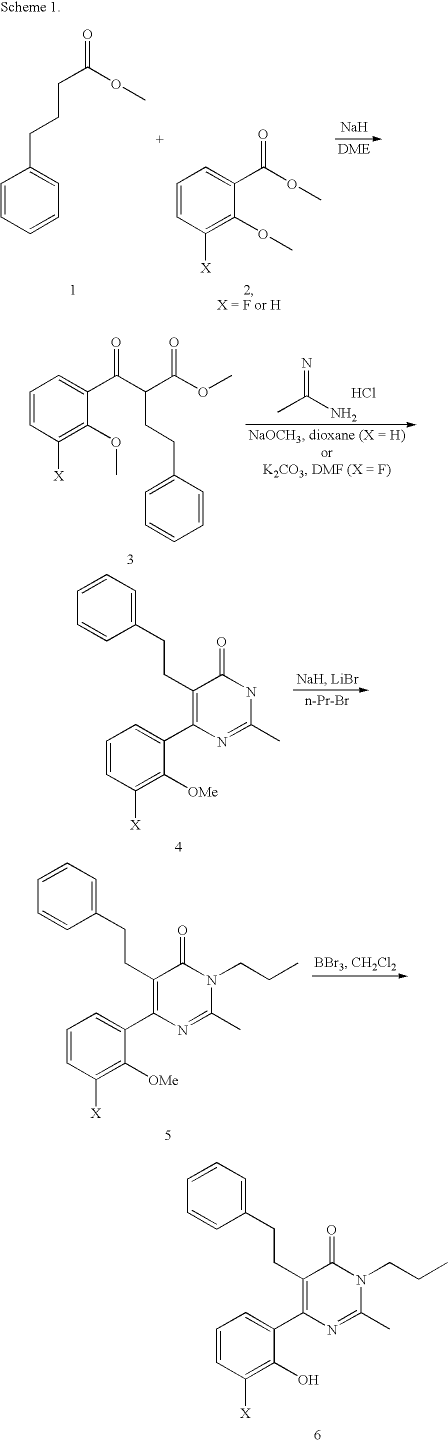

As outlined in Scheme 2, treatment of β-keto ester 3 with sodium acetate in the presence of an acid such as acetic acid provides the enamine 7. Acylation of 7 with an acylating agent such as acetic anhydride provides 8. Treatment of 8 with trimethylaluminum in the presence of an amine such as cyclopropyl amine provides the pyrimidinone 9. Deprotection of 9 under conditions common to the art such as employment of boron tribromide provides the target pyrimidinone 10.

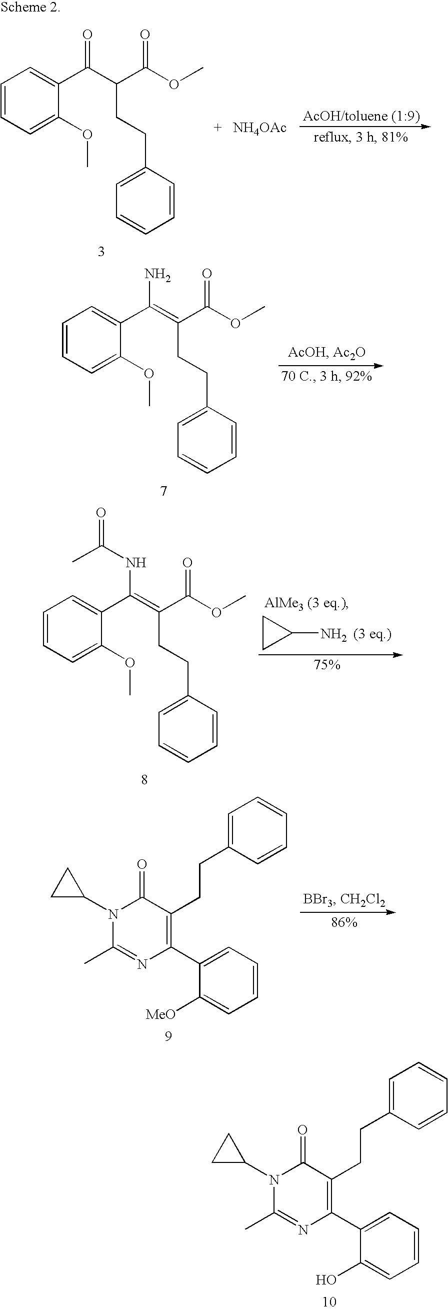

As depicted in Scheme 3, an acetyl-protected enamine such as 11 which can be prepared under the conditions described in Scheme 2 can be treated with chlorotriisopropoxytitanium in the presence of an amine such as 4-aminobiphenyl to induce cyclization to the pyrimidinone 12 in one step. Deprotection of 12 under conditions common to the art such as hydrobromic acid in acetic acid and water provides the target pyrimidinone 13.

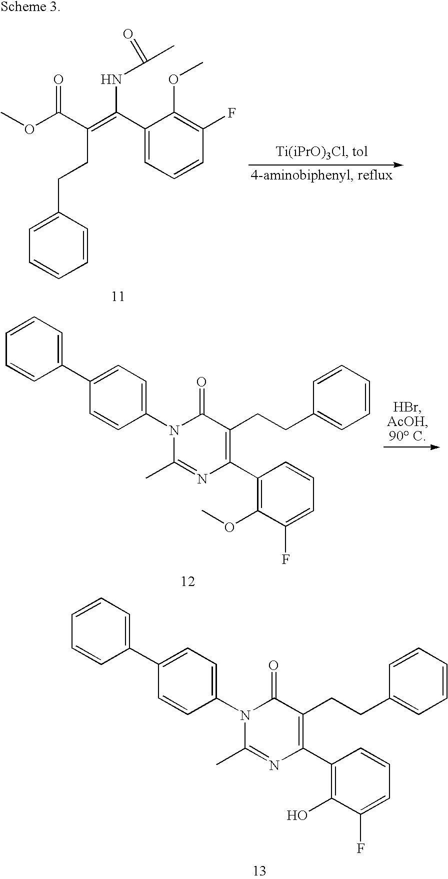

Scheme 4 outlines the route to 2-aminothiophene beginning with commercially available thiophene-2-carboxylate 14. Treatment of carboxylic acid 14 under standard Curtius rearrangement conditions such as diphenylphosphoryl azide in the presence of refluxing tert-butyl alcohol provides the tert-butoxycarbonyl-protected amine 15. Removal of the tert-butylcarbonyl group under conditions common to the art such as trifluoroacetic acid in an organic solvent such as dichloromethane provides the target amine 16.

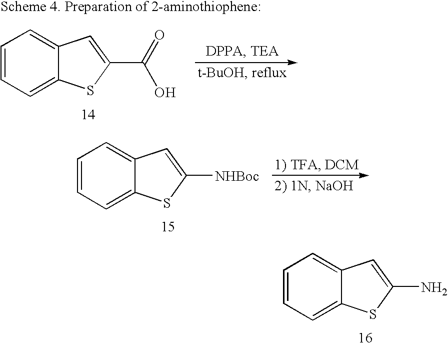

As shown in Scheme 5, treatment of bromide 17 under standard organometallic coupling reaction conditions such as palladium(0) in the presence of an appropriate ligand such as tris(dibenzylidineacetone) and a base such as sodium tert-butoxide in toluene facilitiates coupling between the bromide and an amine such as 1,1-diphenylmethane imine to give imine 18. Standard hydrolysis conditions such as aqueous hydrochloric acid in an organic solvent such as tetrahydrofuran can be employed to provide the free amine target 19.

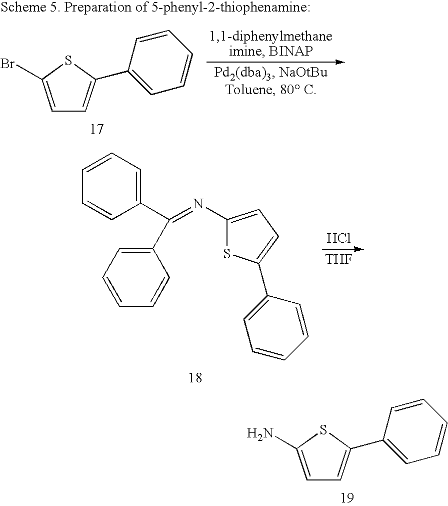

In order to use a compound of Formula (I) or a pharmaceutically acceptable salt thereof for the treatment of humans and other mammals, it is normally formulated in accordance with standard pharmaceutical practice as a pharmaceutical composition.

The calcilytic compounds can be administered by different routes including intravenous, intraperitoneal, subcutaneous, intramuscular, oral, topical (transdermal), or transmucosal administration. Oral administration is suitable for systemic administration. For oral administration, the compounds can be formulated into conventional oral dosage forms. Examples of suitable oral dosage forms include capsules, tablets, and liquid preparations such as syrups, elixirs, and concentrated drops.

Injection (parenteral administration) may also be used, e.g., intramuscular, intravenous, intraperitoneal, and subcutaneous. For injection, the compounds of the invention are formulated in liquid solutions. For example, the compounds may be formulated in physiologically compatible buffers or solutions, such as saline solution, Hank's solution, or Ringer's solution. In addition, the compounds may be formulated in solid form and redissolved or suspended immediately prior to use. Lyophilized forms can also be produced.

Systemic administration can also be achieved by transmucosal or transdermal administration. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays, rectal suppositories, or vaginal suppositories.

For topical administration, the compounds of the invention can be formulated into ointments, salves, gels, or creams, as is generally known in the art.

The amounts of various calcilytic compounds to be administered can be determined by standard procedures taking into account factors such as the compound IC₅₀, EC₅₀, the biological half-life of the compound, the age, size and weight of the patient, and the disease or disorder associated with the patient. The importance of these and other factors to be considered are known to those of ordinary skill in the art.

Amounts administered also depend on the routes of administration and the degree of oral bioavailability. For example, for compounds with low oral bioavailability, relatively higher doses will have to be administered.

The composition is typically administered in unit dosage form. For oral application, for example, a tablet, or capsule may be administered, for nasal application, a metered aerosol dose may be administered, for transdermal application, a topical formulation or patch may be administered and for transmucosal delivery, a buccal patch may be administered. In each case, dosing is such that the patient may administer a single dose.

Each dosage unit for oral administration contains suitably from about 0.01 to about 500 mg/kg of a compound of Formula (I) or a pharmaceutically acceptable salt thereof, calculated as the free base. The dosage unit for oral administration may also be about 0.1 to about 50 mg/kg. The daily dosage for parenteral, nasal, oral inhalation, transmucosal or transdermal routes contains suitably from 0.01 mg to 100 mg/kg, of a compound of Formula (I). A topical formulation contains suitably 0.01 to 5.0% of a compound of Formula (I). While a single does is convenient, multiples doses, such as 2 to 6 times per day may be utilized. As is readily apparent to one skilled in the art, the amount and dosage of the active ingredient may be administered as needed to exhibit the desired activity.

As used herein, “treatment” of a disease includes, but is not limited to prevention, retardation and prophylaxis of the disease.

Diseases and disorders which might be treated or prevented, based upon the affected cells, include bone and mineral-related diseases or disorders; hypoparathyroidism; those of the central nervous system such as seizures, stroke, head trauma, spinal cord injury, hypoxia-induced nerve cell damage, such as occurs in cardiac arrest or neonatal distress, epilepsy, neurodegenerative diseases such as Alzheimer's disease, Huntington's disease and Parkinson's disease, dementia, muscle tension, depression, anxiety, panic disorder, obsessive-compulsive disorder, post-traumatic stress disorder, schizophrenia, neuroleptic malignant syndrome, and Tourette's syndrome; diseases involving excess water reabsorption by the kidney, such as syndrome of inappropriate ADH secretion (SIADH), cirrhosis, congestive heart failure, and nephrosis; hypertension; preventing and/or decreasing renal toxicity from cationic antibiotics (e.g., aminoglycoside antibiotics); gut motility disorders such as diarrhea and spastic colon; GI ulcer diseases; GI diseases with excessive calcium absorption such as sarcoidosis; autoimmune diseases and organ transplant rejection; squamous cell carcinoma; and pancreatitis.

In one embodiment, the reversed pyrimidinone compounds are used to increase serum parathyroid hormone (“PTH”) levels. Increasing serum PTH levels can be helpful in treating diseases such as hypoparathyroidism, osteosarcoma, periodontal disease, fracture, osteoarthritis, rheumatoid arthritis, Paget's disease, humoral hypercalcemia malignancy and osteoporosis.

The reversed pyrimidinone compounds can be co-administered with an anti-resorptive agent. Such agents include, but are not limited estrogen, 1, 25 (OH)₂ vitamin D3, calcitonin, selective estrogen receptor modulators, vitronectin receptor antagonists, V−H+-ATPase inhibitors, src SH2 antagonists, bisphosphonates and cathepsin K inhibitors.

The compounds disclosed herein can be utilized in a method of treating a patient to increase the patient's serum PTH level. The method is carried out by administering to the patient an amount of the compound effective to cause an increase in duration and/or quantity of serum PTH level sufficient to have a therapeutic effect.

In various embodiments, the compound administered to a patient causes an increase in serum PTH having a duration of up to one hour, about one to about twenty-four hours, about one to about twelve hours, about one to about six hours, about one to about five hours, about one to about four hours, about two to about five hours, about two to about four hours, or about three to about six hours.

In another embodiment, the compound administered to a patient causes an increase in serum PTH having a duration of more than about twenty four hours provided that it is co-administered with an anti resorptive agent.

In additional different embodiments, the compound administered to a patient causes an increase in serum PTH of up to two fold, two to five fold, five to ten fold, and at least 10 fold, greater than peak serum PTH in the patient. The peak serum level is measured with respect to a patient not undergoing treatment.

As indicated above, compounds of Formula (I) and their pharmaceutically acceptable salts, which are active when given orally, can be formulated as syrups, tablets, capsules and lozenges. A syrup formulation generally comprises a suspension or solution of the compound or salt in a liquid carrier. Examples of suitable liquid carriers include ethanol, peanut oil, olive oil, glycerine or water with a flavoring or coloring agent. In a composition provided in the form of a tablet, any pharmaceutical carrier routinely used for preparing solid formulations may be used. Examples of such carriers include magnesium stearate, terra alba, talc, gelatin, acacia, stearic acid, starch, lactose and sucrose. For a compound provided in a capsule, any routine encapsulation is suitable. For example, the aforementioned carriers used in preparing tablets may be utilized to form a hard gelatin capsule shell. For compositions in a soft gelatin shell capsule, any pharmaceutical carrier routinely used for preparing dispersions or suspensions may be considered. Examples of suitable materials for forming a soft gelatin capsule shell include aqueous gums, celluloses, silicates and oils.

Typical parenteral compositions comprise a solution or suspension of a compound or salt in a sterile aqueous or non-aqueous carrier optionally containing parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of Formula (I) or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

The composition is conveniently provided in unit dosage form, for example a tablet, capsule or metered aerosol dose, so that the patient may administer a single dose.

Additional information about standard pharmaceutical practice for formulating pharmaceutical compositions such as conventional techniques for making tablets and pills containing active ingredients are described in the standard reference, “Remington: the Science and Practice of Pharmacy,” (21st ed. 2005). This standard reference is incorporated herein.

No unacceptable toxological effects are expected when reversed pyrimidinone compounds are administered in accordance with the understanding of one of ordinary skill in the art.

### EXAMPLES OF PREPARING REVERSED PYRIMIDINONES

The following specific examples are included for illustrative purposes only and are not to be considered as limiting to this disclosure. The reagents and intermediates used in the following examples are either commercially available or can be prepared according to standard literature procedures by those skilled in the art of organic synthesis.

### Example 1

### Preparation of 6-(2-hydroxyphenyl)-2-methyl-5-(2-phenylethyl)-3-propyl-4(3H)-pyrimidinone

### a. 2-[1-(2-Methoxy-phenyl)-methanoyl]-4-phenyl-butyric acid methyl ester

To a suspension of sodium hydride (4.31 g, 179.6 mmol) in DME (100 ml) at 0° C. was slowly added methyl 4-phenyl butyrate (8.0 g, 44.89 mmol). After stirring for 15 minutes, methyl 2-(methoxy)benzoate (9.67 ml, 67.32 mmol) was added, followed with addition of 8 drops of methanol. The reaction mixture was heated to reflux for 3 hrs, cooled in an ice-bath, quenched carefully with 1N HCl, and extracted with ethyl ether (150 mL×3). The organic layers were combined and dried over MgSO₄, filtered, and purified by silica gel chromatography (0-4%, ethyl acetate/hexane) to provide the title compound (10.5 g, 75%). LCMS (m/z): 313 (M+H).

### b. 2-methyl-6-[2-(methyloxy)phenyl]-5-(2-phenylethyl)-4(1H)-pyrimidinone

To the solution of acetamidine (1.51 g, 15.98 mmol) in methanol/dioxane (130 mL/26 mL) was added NaOCH₃ (6.5 mL, 25% w/w in methanol). After stirring the mixture for 5-10 minutes, methyl 2-{[2-(methyloxy)phenyl]carbonyl}-4-phenylbutanoate (from a) (2.77 g, 8.88 mmol) was added, and the reaction mixture was heated to reflux for 24 hours. After heating the reaction mixture overnight some starting material was still present. Additional acetamidine (252 mg, 2.66 mmol) and NaOCH₃ (1.22 ml, 25% w/w in methanol) were added and heating was continued for several hours. Finally, the solvent was evaporated, and the residue was diluted with water. The pH of the mixture was adjusted to ˜7-8 using acetic acid followed by extraction with dichloromethane (100 mL×3). The organic layers were dried over MgSO₄ and evaporated under reduced pressure. Purification of the residue using silica gel chromatography (methanol/dichloromethane, 0-1.5%) provided the title compound (1.62 g, 64%). LCMS (m/z): 321 (M+H).

### c. 2-methyl-6-[2-(methyloxy)phenyl]-5-(2-phenylethyl)-3-propyl-4(3H)-pyrimidinone

To a stirred solution of 2-methyl-6-[2-(methyloxy)phenyl]-5-(2-phenylethyl)-4(1H)-pyrimidinone (from b) (209 mg, 0.653 mmol) in DMF at RT was added sodium hydride (52 mg, 1.31 mmol, 60% dispersion in mineral oil) under argon. After stirring the reaction mixture for about two minutes, anhydrous LiBr (170 mg, 1.96 mmol) was added, and the stirring was continued for a few minutes before addition of 1-bromo-propane (0.098 mL, 1.08 mmol). The reaction mixture was stirred overnight, and the solvent was then evaporated under reduced pressure. The residue was diluted with DCM, washed sequentially with water and brine then dried over MgSO₄. The organic phase was then concentrated and purified by silica chromatography (10-20%, ethyl acetate/hexanes) to provide the title compound (140 mg, 59%). LCMS (m/z): 363 (M+H).

### d. 6-(2-hydroxyphenyl)-2-methyl-5-(2-phenylethyl)-3-propyl-4(3H)-pyrimidinone

To a stirred solution of 2-methyl-6-[2-(methyloxy)phenyl]-5-(2-phenylethyl)-3-propyl-4(3H)-pyrimidinone (140 mg, 0.39 mmol) in CH₂Cl₂ (4.0 mL) at −40° C. was added BBr₃ (1.55 mL, 1.55 mmol, 1M in CH₂Cl₂). The temperature of the reaction mixture was raised to 0° C. and the stirring was continued for 2 hr. The reaction mixture was then quenched by pouring it into an ice-cold saturated aq. NaHCO₃ solution. The mixture was extracted with dichloromethane (50 mL×2) and the organic layer was washed with brine and dried over MgSO₄. After evaporation under reduced pressure, the residue was purified by silica gel chromatography (5-65%, ethyl acetate/hexanes) to provide the title compound (89 mg, 66%): ¹H NMR (400 MHz, CDCl₃): δ 7.37-7.18 (m, 7H), 7.06 (d, 1H), 6.95 (t, 1H), 4.05 (m, 2H), 2.91 (s, 4H), 2.70 (s, 3H), 2.10 (m, 2H), 1.08 (t, 3H); LCMS (m/z): 349.4 (M+H).

### Example 2

### Preparation of 6-(2-hydroxyphenyl)-2-methyl-5-(2-phenylethyl)-3-ethyl-4(3H)-pyrimidinone

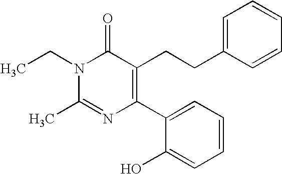

The title compound was prepared following the general procedures of Example 1 except substituting 1-bromoethane for 1-bromopropane: ¹H NMR (400 MHz, CDCl₃) δ7.25-7.02 (m, 7H), 6.93 (d, 1H), 6.88 (t, 1H), 4.05 (q, 2H), 2.85 (s, 4H), 2.62 (s, 3H), 1.28 (t, 3H); LCMS (m/z) 335.2 (M+H).

### Example 3

### Preparation of 6-(2-hydroxyphenyl)-2-methyl-5-(2-phenylethyl)-3-methyl-4(3H)-pyrimidinone

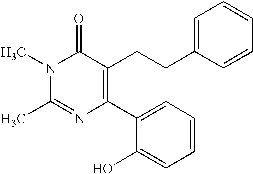

The title compound was prepared following the general procedures of Example 1 except substituting 1-bromomethane for 1-bromopropane: ¹H NMR (400 MHz, CDCl₃) δ 7.32-7.14 (m, 7H), 7.03 (d, 1H), 6.91 (t, 1H), 3.62 (s, 3H), 2.96 (s, 4H), 2.71 (s, 3H); LCMS (m/z): 321.2 (M+H).

### Example 4

### Preparation of 6-(2-hydroxyphenyl)-2-methyl-5-(2-phenylethyl)-3-[2-(2-pyridinyl)ethyl]-4(3H)-pyrimidinone

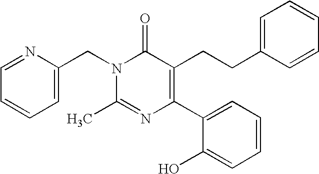

The title compound was prepared following the general procedures of Example 1 except substituting 1-bromomethylpyridine for 1-bromopropane: ¹H NMR (400 MHz, CDCl₃) δ 8.63 (d, 1H), 7.86 (t, 1H), 7.42-7.19 (m, 9H), 7.08 (d, 1H), 6.95 (t, 1H), 5.44 (s, 2H), 3.02 (s, 4H), 2.78 (s, 3H), 1.66 (br, 1H); LCMS (m/z): 398.2 (M+H).

### Example 5

### Preparation of 6-(2-hydroxyphenyl)-2-methyl-5-(2-phenylethyl)-3-butyl-4(3H)-pyrimidinone

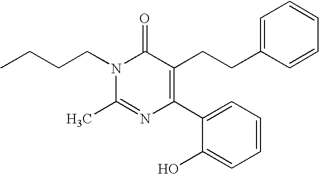

The title compound was prepared following the general procedures of Example 1 except substituting 1-bromobutane for 1-bromopropane: ¹H NMR (400 MHz, CDCl₃): δ 7.36-7.18 (m, 7H), 7.06 (d, 1H), 6.95 (t, 1H), 4.10 (m, 2H), 3.90 (s, 4H), 2.69 (s, 3H), 1.74 (m, 2H), 1.48 (m, 2H), 0.91 (t, 3H); LCMS (m/z): 363.2 (M+H).

### Example 6

### Preparation of 6-(2-hydroxyphenyl)-2-methyl-5-(2-phenylethyl)-3-pentyl-4(3H)-pyrimidinone

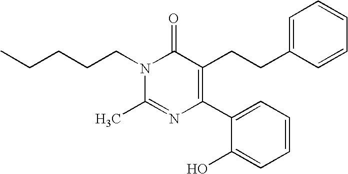

The title compound was prepared following the general procedures of Example 1 except substituting 1-bromopentane for 1-bromopropane: ¹H NMR (400 MHz, CDCl₃) δ 7.31-7.21 (m, 7H), 7.09 (d, 1), 6.92 (t, 1H), 3.98 (m, 2H), 2.87 (s, 4H), 2.57 (s, 3H), 1.69 (m, 2H), 1.35 (m, 4H), 0.87 (m, 3H); LCMS (m/z): 377.4 (M+H).

### Example 7

### Preparation of 6-(2-hydroxy-phenyl)-2-methyl-5-(2-phenethyl)-3-hexyl-3H-pyrimidin-4-one

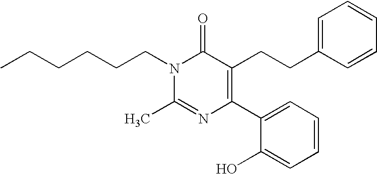

The title compound was prepared following the general procedures of Example 1 except substituting 1-bromohexane for 1-bromopropane: ¹H NMR (400 MHz, CDCl₃) δ 7.36-7.19 (m, 7H), 7.05 (d, 1H), 6.96 (t, 1H), 4.07 (m, 2H), 2.95 (s, 4H), 2.57 (s, 3H), 1.78 (m, 2H), 1.52-0.92 (m, 9H); LCMS (m/z): 391.4 (M+H).

### Example 8

### Preparation of 3-cyclopropylmethyl-6-(2-hydroxy-phenyl)-2-methyl-5-phenethyl-3H-pyrimidin-4-one

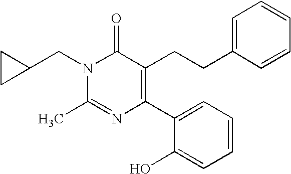

The title compound was prepared following the general procedures of Example 1 except substituting bromocyclopropylmethane for 1-bromopropane: ¹H NMR (400 MHz, CDCl₃) δ 7.30-7.09 (m, 7H), 7.01 (d, 1H), 6.98 (t, 1H), 4.07 (d, 2H), 2.96 (s, 4H), 2.77 (s, 3H), 1.22 (m, 1H), 0.68 (m, 2H), 0.55 (m, 2H); LCMS (m/z): 361.2 (M+H).

### Example 9

### Preparation of 3-(2-methylallyl)-6-(2-hydroxy-phenyl)-2-methyl-5-phenethyl-3H-pyrimidin-4-one

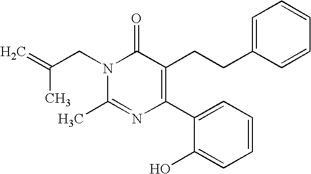

The title compound was prepared following the general procedures of Example 1 except substituting 3-bromo-2-methylpropene for 1-bromopropane: ¹H NMR (400 MHz, CDCl₃) δ 7.39-7.21 (m, 7H), 7.03 (d, 1H), 7.20 (t, 1H), 5.00 (s, 1H), 4.68 (s, 2H), 4.57 (s, 1H), 2.98 (s, 4H), 2.60 (s, 3H), 2.02 (s, 3H); LCMS (m/z): 361.2 (M+H).

### Example 10

### Preparation of 3-(3-methyl butyl)-6-(2-hydroxy-phenyl)-2-methyl-5-phenethyl-3H-pyrimidin-4-one

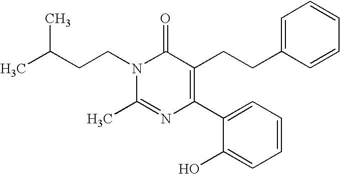

The title compound was prepared following the general procedures of Example 1 except substituting 1-bromo-3-methylbutane for 1-bromopropane: ¹H NMR (400 MHz, CDCl₃) δ 10.30 (br, 1H, OH), 7.41-6.92 (m, 9H), 4.08 (t, 2H), 2.97 (m, 4H), 2.64 (s, 3H), 1.80 (m, 1H), 1.69-1.63 (m, 2H), 1.06 (d, 6H); LCMS (m/z) 377.2 (M+H).

### Example 11

### Preparation of 3-(2-cyclohexylethyl)-6-(2-hydroxy-phenyl)-2-methyl-5-phenethyl-3H-pyrimidin-4-one

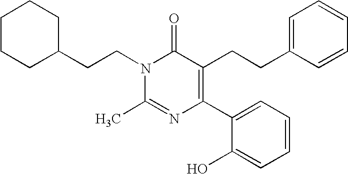

The title compound was prepared following the general procedures of Example 1 except substituting 1-bromo-2-cyclohexylethane for 1-bromopropane: ¹H NMR (400 MHz, CDCl₃) δ 10.20 (br, 1H, OH), 7.43-6.92 (m, 9H), 4.09 (t, 2H), 2.98 (s, 4H), 2.62 (s, 3H), 1.32-1.06 (m, 13H); LCMS (m/z): 417.4 (M+H).

### Example 12

### Preparation of 3-propyl-6-(3-fluoro-2-hydroxy-phenyl)-2-methyl-5-phenethyl-3H-pyrimidin-4-one

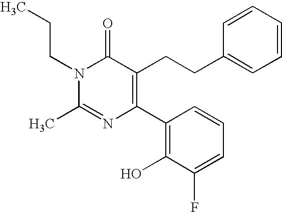

The title compound was prepared following the general procedures of Example 1 except substituting 3-fluoro-2-methoxybenzoic acid for 3-methoxybenzoic acid: ¹H NMR (400 MHz, CDCl₃): δ 10.25 (br, 1H), 7.30-7.12 (m, 7H), 6.81 (m, 1H), 4.02 (m, 2H), 2.92 (s, 4H), 2.59 (s, 3H), 1.76 (m, 2H), 1.41 (m, 6H), 0.95 (t, 3H); LCMS (m/z): 367.2 (M+H).

### Example 13

### Preparation of 3-hexyl-6-(3-fluoro-2-hydroxy-phenyl)-2-methyl-5-phenethyl-3H-pyrimidin-4-one

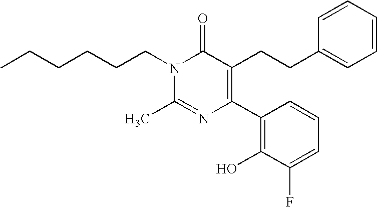

The title compound was prepared following the general procedures of Example 1 except substituting 3-fluoro-2-methoxybenzoic acid for 3-methoxybenzoic acid: ¹H NMR (400 MHz, CDCl₃): δ 10.25 (br, 1H), 7.30-7.10 (m, 7H), 6.88 (m, 1H), 4.06 (m, 2H), 2.99 (s, 4H), 2.61 (s, 3H), 1.82 (m, 2H), 1.07 (t, 3H); LCMS (m/z): 409.2 (M+H).

### Example 14

### Preparation of 3-propyl-6-(2-hydroxy-phenyl)-2-methyl-5-(2-cyclohexylethyl)-3H-pyrimidin-4-one

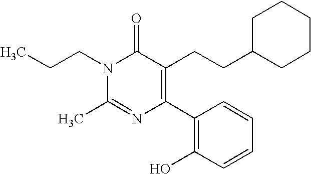

The title compound was prepared following the general procedures of Example 1 except substituting methyl-4-cylcobutyrate for 4-phenylbutyrate: ¹H NMR (400 MHz, CDCl₃) δ 10.50 (s, 1H, OH), 7.40 (d, 1H), 7.37-7.28 (t, 1H), 7.02 (d, 1H), 6.91 (t, 1H), 4.00 (t, 2H), 2.65 (t, 2H), 2.60 (s, 3H), 1.88-1.52 (m, 9H), 1.39-0.90 (m, 9H); LCMS (m/z): 355.4 (M+H).

### Example 15

### Preparation of 2-(2-hydroxyphenyl)-3-(2-phenylethyl)-6,7,8,9-tetrahydro-4H-pyrido[1,2-a]pyrimidin-4-one

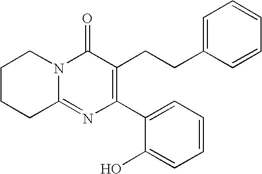

The title compound was prepared following the general procedures of Example 1 except substituting acetamidine with 3,4,5,6-tetrahydro-2-pyridineamine ¹H NMR (400 MHz, CDCl₃) δ7.15-7.46 (m, 7H), 7.06 (d, 1H), 6.90 (t, 1H), 4.11 (t, 2H), 2.93-3.11 (m, 4H), 1.90-2.18 (m, 4H), 1.50-1.68 (m, 2H); LCMS (m/z): 347.3 (M+H).

### Example 16

### Preparation of 3-(2-cyclohexylethyl)-2-(2-hydroxyphenyl)-6,7,8,9-tetrahydro-4H-pyrido[1,2-a]pyrimidin-4-one

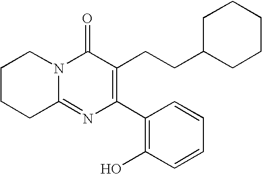

The compound was prepared by substituting piperidin-2-ylideneamine for acetamidine in Example 1b and methyl 4-cyclohexylbutyrate for methyl 4-phenylbutyrate in Example 1a: ¹H NMR (400 MHz, CDCl₃), 10.50 (br, 1H, OH), 7.41 (d, 1H), 7.35-7.28 (t, 1H), 7.00 (d, 1H), 6.91 (t, 1H), 4.00 (t, 2H), 2.92 (t, 2H), 2.70-2.65 (m, 2H), 2.10-1.90 (m, 4H), 1.79-1.55 (m, 9H), 1.48-1.12 (m, 4H), 1.00-0.90 (m, 2H); LCMS (m/z): 353.4 (M+H).

### Example 17

### Preparation of 3-cyclopropyl-6-(2-hydroxyphenyl)-2-methyl-5-(2-phenylethyl)pyrimidin-4(3H)-one

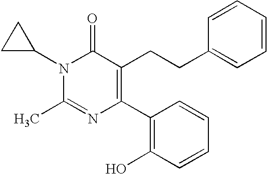

### a. Methyl (2Z)-3-amino-3-[2-(methyloxy)phenyl]-2-(2-phenylethyl)-2-propenoate

To a solution of methyl 2-{[2-(methyloxy)phenyl]carbonyl}-4-phenylbutanoate (2.0 g, 6.4 mmol) in toluene (12 mL) were added ammonium acetate (3.0 g, 38.5 mmol) and 1.3 mL acetic acid at rt. The reaction vessel was fitted with a Dean-Stark trap and a condenser and then was heated to reflux for 3 hrs. The reaction mixture was cooled to room temperature, concentrated, and the crude mixture was used in the next step without further purification. LCMS (m/z): 311.3 (M+H).

### b. Methyl (2Z)-3-(acetylamino)-3-[2-(methyloxy)phenyl]-2-(2-phenylethyl)-2-propenoate

To the crude material from Example 17a (1 g, 3.2 mmol) was added acetic anhydride (9 mL) and acetic acid (2 mL). After heating at 70° C. for 3 hrs, the reaction mixture was cooled to room temperature and concentrated. The residue was diluted with saturated NaHCO₃ and then extracted with CH₂Cl₂ twice. The combined organic layers were dried over MgSO₄, filtered, and concentrated. Purification by silica chromatography (5-40% ethyl acetate/hexanes) provided product (0.99 g, 92%). LCMS (m/z): 353.4 (M+H).

### c. 3-Cyclopropyl-2-methyl-6-[2-(methyloxy)phenyl]-5-(2-phenylethyl)-4(3H)-pyrimidinone

To a solution of cyclopropanamine (0.12 mL, 1.7 mmol) in dry CH₂Cl₂ (4 mL) was slowly added 0.85 mL (1.7 mmol) of a 2.0 M solution of Me₃Al in heptane at room temperature under nitrogen. After stirring 20 min, the enamide from Example 17b (0.2 g, 0.59 mmol) was added. The reaction mixture was heated to reflux for 3 hrs, then cooled to room temperature before the quenching by the slow addition of 1N HCl. The resulting mixture was extracted with CH₂Cl₂, and the combined organic layer was washed with saturated NaHCO₃ and brine. After drying over MgSO₄ and concentration in vacuo, silica chromatography (10-60% ethyl acetate/hexanes) afforded the title compound (0.16 g, 75%). LCMS (m/z): 360.4 (M+H).

### d. 3-Cyclopropyl-6-(2-hydroxyphenyl)-2-methyl-5-(2-phenylethyl)-4(3H)-pyrimidinone

To a −60° C. solution of 3-cyclopropyl-2-methyl-6-[2-(methyloxy)phenyl]-5-(2-phenylethyl)-4(3H)-pyrimidinone (0.16 g, 0.44 mmol) in 3 mL CH₂Cl₂ under a dry nitrogen atmosphere was slowly added BBr₃ (2.83 mL, 1M solution in CH₂Cl₂). The reaction mixture was warmed to 0° C. and stirred for 3 hrs. The reaction was quenched by the addition of 1:1 (H₂O:sat. NaHCO₃), extracted with CH₂Cl₂ three times, and the combined organic layer was dried over MgSO₄, filtered and concentrated. The resultant residue was purified by silica chromatography (0.2-0.8% MeOH/CH₂Cl₂) to provide the desired product (132 mg, 86%): ¹H NMR (400 MHz, CDCl₃): δ 7.21-7.51 (m, 7H), 7.04 (d, 1H), 6.95 (t, 1H), 2.96-3.12 (m, 1H), 2.90-3.09 (m, 4H), 3.71 (s, 3H), 1.38-1.41 (m, 2H), 0.98-1.07 (m, 2H); LCMS (m/z): 347.25 (M+H).

### Example 18

### Preparation of 6-(2-Hydroxyphenyl)-2-methyl-3-[2-(1-methylpyrrolidin-2-yl)ethyl]-5-(2-phenylethyl)pyrimidin-4(3H)-one

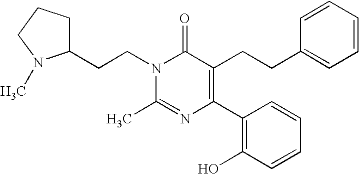

The title compound was prepared following the general procedures of Example 17 except substituting cyclopropanamine with [2-(1-methyl-2-pyrrolidinyl)ethyl]amine: ¹H NMR (400 MHz, CDCl₃): δ 7.12-7.51 (m, 7H), 7.04 (d, 1H), 6.95 (t, 1H), 4.20-4.28 (m, 1H), 4.01-4.11 (m, 1H), 3.21-3.32 (m, 1H), 2.94-3.05 (m, 4H), 2.65 (s, 3H), 2.51 (s, 3H), 2.32-2.41 (m, 1H), 2.11-2.24 (m, 2H), 1.70-1.95 (m, 4H), 0.81-0.91 (m, 1H); LCMS (m/z): 418.2 (M+H).

### Example 19

### Preparation of 3-(2,2-dimethylpropyl)-6-(2-hydroxyphenyl)-2-methyl-5-(2-phenylethyl)pyrimidin-4(3H)-one

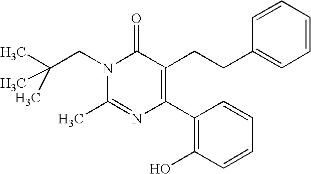

The title compound was prepared following the general procedures of Example 17 except substituting cyclopropanamine with (2,2-dimethylpropyl)amine ¹H NMR (400 MHz, CDCl₃): δ 7.18-7.45 (m, 7H), 7.04 (d, 1H), 6.91 (t, 1H), 4.10-4.15 (m, 2H), 2.92-3.02 (m, 4H), 2.62 (s, 3H), 1.08 (s, 9H); LCMS (m/z): 377.6 (M+H).

### Example 20

### Preparation of 3-sec-butyl-6-(2-hydroxyphenyl)-2-methyl-5-(2-phenylethyl)pyrimidin-4(3H)-one

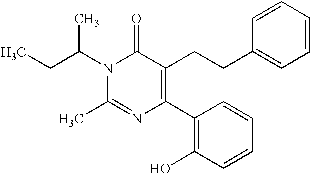

The title compound was prepared following the general procedures of Example 17 except substituting cyclopropanamine with 2-butanamine: ¹H NMR (400 MHz, CDCl₃): δ 6.98-7.22-7.5 (m, 7H), 6.88 (d, 1H), 6.72 (t, 1H), 3.95-4.07 (m, 1H), 2.72-2.81 (m, 4H), 2.42 (s, 3H), 2.12-2.20 (m, 1H), 1.71-1.89 (m, 1H), 1.45 (d, 2H), 0.71 (t, 3H); LCMS (m/z): 363.3 (M+H).

### Example 21

### Preparation of 3-cyclopentyl-6-(2-hydroxyphenyl)-2-methyl-5-(2-phenylethyl)pyrimidin-4(3H)-one

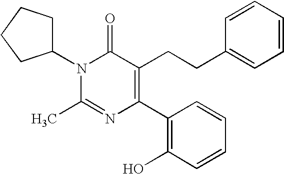

The title compound was prepared following the general procedures of Example 17 except substituting cyclopropanamine with cyclopentanamine: ¹H NMR (400 MHz, CDCl₃): δ 7.15-7.43 (m, 7H), 7.05 (d, 1H), 6.95 (t, 1H), 4.61-4.72 (m, 1H), 2.92-3.03 (m, 4H), 2.65 (s, 3H), 2.38-2.86 (m, 2H), 2.14-2.20 (m, 2H), 1.92-1.99 (m, 2H), 1.63-1.73 (m, 2H); LCMS (m/z): 375.2 (M+H).

### Example 22

### Preparation of 6-(2-hydroxyphenyl)-3-isobutyl-2-methyl-5-(2-phenylethyl)pyrimidin-4(3H)-one

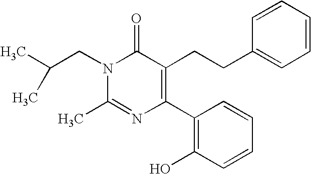

The title compound was prepared following the general procedures of Example 17 except substituting cyclopropanamine with (2-methylpropyl)amine: ¹H NMR (400 MHz, CDCl₃): δ 7.15-7.48 (m, 7H), 7.02 (d, 1H), 6.88 (t, 1H), 3.92 (d, 2H), 2.85-2.96 (m, 4H), 2.55 (s, 3H), 1.01 (d, 6H); LCMS (m/z): 363.4 (M+H).

### Example 23

### Preparation of 3-cyclobutyl-6-(2-hydroxyphenyl)-2-methyl-5-(2-phenylethyl)pyrimidin-4(3H)-one

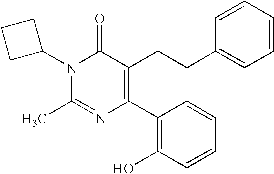

The title compound was prepared following the general procedures of Example 17 except substituting cyclopropanamine with cyclobutanamine: ¹H NMR (400 MHz, CDCl₃): δ 7.18-7.50 (m, 7H), 7.04 (d, 1H), 6.91 (t, 1H), 4.67-4.80 (m, 1H), 3.04-3.15 (m, 2H), 2.94-2.99 (m, 4H), 2.40-2.52 (m, 2H), 2.00-2.10 (m, 1H), 1.80-1.91 (m, 1H); LCMS (m/z): 361.5 (M+H).

### Example 24

### Preparation of 3-cyclohexyl-6-(2-hydroxyphenyl)-2-methyl-5-(2-phenylethyl)pyrimidin-4(3H)-one

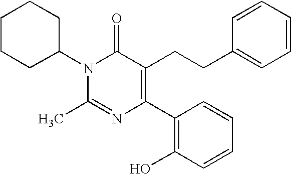

The title compound was prepared following the general procedures of Example 17 except substituting cyclopropanamine with cyclohexanamine: ¹H NMR (400 MHz, CDCl₃): δ 7.12-7.54 (m, 7H), 7.01 (d, 1H), 6.89 (t, 1H), 4.01-4.21 (m, 1H), 2.89-3.02 (m, 4H), 2.62 (s, 3H), 1.92-2.00 (m, 2H), 1.71-1.82 (m, 4H), 1.32-1.45 (m, 4H); LCMS (m/z): 389.4 (M+H).

### Example 25

### Preparation of 6-(2-hydroxyphenyl)-3-isopropyl-2-methyl-5-(2-phenylethyl)pyrimidin-4(3H)-one

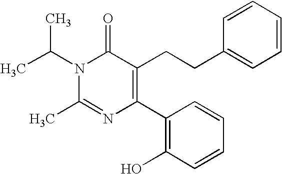

The title compound was prepared following the general procedures of Example 17 except substituting cyclopropanamine with 2-propanamine: ¹H NMR (400 MHz, CDCl₃): δ 7.08-7.29 (m, 7H), 6.90 (d, 1H), 6.82 (t, 1H), 4.45-4.52 (m, 1H), 2.85-2.96 (m, 4H), 2.53 (s, 3H), 1.59 (s, 6H); LCMS (m/z): 349.5 (M+H).

### Example 26

### Preparation of 6-(2-hydroxyphenyl)-2-methyl-5-(2-phenylethyl)-3-(2,2,2-trifluoroethyl)pyrimidin-4(3H)-one

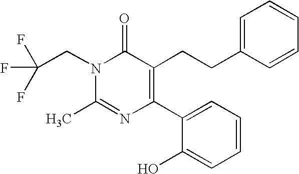

The title compound was prepared following the general procedures of Example 17 except substituting cyclopropanamine for 1,1,1-trifluoropropylamine: ¹H NMR (400 MHz, CDCl₃): δ 7.18-7.45 (m, 7H), 7.06 (d, 1H), 6.91 (t, 1H), 4.79-4.85 (m, 2H), 2.95-3.02 (m, 4H), 2.63 (s, 3H), 1.55 (s, 2H); LCMS (m/z): 389.2 (M+H).

### Example 27

### Preparation of 6-(2-hydroxyphenyl)-2-methyl-3-octyl-5-(2-phenylethyl)pyrimidin-4(3H)-one

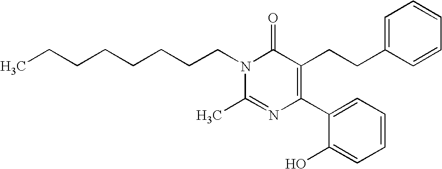

The title compound was prepared following the general procedures of Example 1 except substituting 1-bromoethane with 1-iodooctane: ¹H NMR (400 MHz, CDCl₃) δ7.15-7.55 (m, 7H), 7.01 (d, 1H), 6.89 (t, 1H), 3.97-4.10 (m, 2H), 2.96-3.08 (m, 4H), 2.62 (s, 3H), 1.70-1.75 (m, 2H), 1.12-1.45 (m, 10H), 0.91 (t, 3H); LCMS (m/z): 419.4 (M+H).

### Example 28

### Preparation of 3-heptyl-6-(2-hydroxyphenyl)-2-methyl-5-(2-phenylethyl)pyrimidin-4(3H)-one

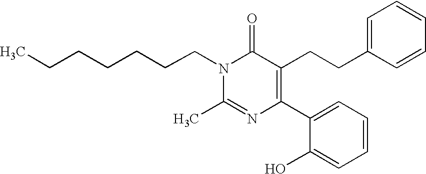

The title compound was prepared following the general procedures of Example 1 except substituting 1-bromoethane with 1-bromoheptane: ¹H NMR (400 MHz, CDCl₃) δ7.15-7.46 (m, 7H), 6.99 (d, 1H), 6.88 (t, 1H), 3.92-4.10 (m, 2H), 2.96-3.08 (m, 4H), 2.68 (s, 3H), 1.70-1.75 (m, 2H), 1.18-1.45 (m, 8H), 0.89 (t, 3H); LCMS (m/z): 405.6 (M+H).

### Example 29

### Preparation of 3-allyl-6-(2-hydroxyphenyl)-2-methyl-5-(2-phenylethyl)pyrimidin-4(3H)-one

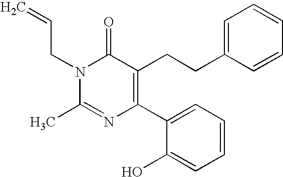

The title compound was prepared following the general procedures of Example 1 except substituting 1-bromoethane with allyl bromide: ¹H NMR (400 MHz, CDCl₃) δ7.15-7.46 (m, 7H), 7.06 (d, 1H), 6.90 (t, 1H), 5.82-6.01 (m, 1H), 5.12-5.45 (m, 2H), 4.75 (s, 1H), 2.95-3.00 (m, 4H), 2.65 (s, 3H); LCMS (m/z): 347.2 (M+H).

### Example 30

### Preparation of 6-(3-fluoro-2-hydroxyphenyl)-2,3-dimethyl-5-(2-phenylethyl)pyrimidin-4(3H)-one

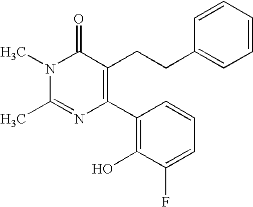

### a. Methyl 3-fluoro-2-(methyloxy)benzoate

To a solution of 3-fluoro-2-hydroxybenzoic acid (100 mg, 0.64 mmol) in DMF under argon, was first added CsCO₃ (0.75 g, 2.24 mmol), and then CH₃I (0.10 mL, 1.6 mmol). This mixture was stirred at room temperature overnight. The DMF was removed in vacuo and the residue was diluted in dichloromethane. The reaction contents were filtered to remove solid, and the filtrate was washed with brine. The title compound (78 mg) was isolated and carried on the next step without further purification. ¹H NMR (400 MHz, CDCl₃) d: 3.90 (s, 3H), 4.00 (s, 3H), 7.10 (m, 1H), 7.30 (m, 1H), 7.60 (m, 1H).

### b. Methyl 2-{[3-fluoro-2-(methyloxy)phenyl]carbonyl}-4-phenylbutanoate

The title compound was prepared according to the general procedure described in Example 1a. ¹H NMR (400 MHz, CDCl₃) δ: 2.26-2.33 (m, 2H), 2.71 (t, 2H) 3.73 (s, 3H), 3.92 (d, 3H), 4.29 (t, 1H), 7.04-7.31 (m, 7H), 7.44 (d, 1H).

### c. 6-[3-fluoro-2-(methyloxy)phenyl]-2-methyl-5-(2-phenylethyl)-4(1H)-pyrimidinone

To the solution of acetamidine (370 mg, 3.91 mmol) in DMF was added K₂CO₃ (1.24 g, 7.8 mmol), and the resulting suspension was stirred for 5-10 min. Methyl 2-{[3-fluoro-2-(methyloxy)phenyl]carbonyl}-4-phenylbutanoate from Example 30b (530 mg, 1.56 mmol) was added to the reaction vessel, and the resulting mixture was heated to reflux for 24 hours. The reaction mixture was cooled to room temperature and then poured into 110 mL H₂O. The pH was adjusted to 3-4 by 1N HCl, and the mixture was extracted with EtOAc (2×). The organic layer was dried, filtered and concentrated. Purification of the residue by silica chromatography (0-3% MeOH/DCM) provided 400 mg of the product (75%). LCMS (m/z): 353.2 (M+H).

### d. 6-(3-fluoro-2-hydroxyphenyl)-2,3-dimethyl-5-(2-phenylethyl)pyrimidin-4(3H)-one

The title compound was prepared according to the general procedures described in Examples 1c and 1d. ¹H NMR (400 MHz, CDCl₃): δ 10.11 (s, 1H), 6.82-7.28 (m, 8H), 3.65 (s, 3H), 2.85-3.01 (m, 4H), 2.65 (s, 3H); LCMS (m/z): 339.3 (M+H).

### Example 31

### Preparation of 3-ethyl-6-(3-fluoro-2-hydroxyphenyl)-2-methyl-5-(2-phenylethyl)pyrimidin-4(3H)-one

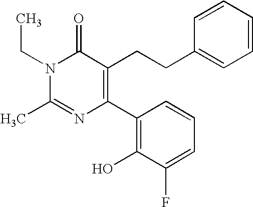

The title compound was prepared following the general procedures of Example 30 except substituting iodomethane with bromoethane: ¹H NMR (400 MHz, CDCl₃) δ10.15 (s, 1H), 6.78-7.21 (m, 8H), 4.15 (q, 2H), 2.88-2.98 (m, 4H), 2.62 (s, 3H), 1.38 (t, 3H); LCMS (m/z): 353.2 (M+H).

### Example 32

### Preparation of 3-butyl-6-(3-fluoro-2-hydroxyphenyl)-2-methyl-5-(2-phenylethyl)pyrimidin-4(3H)-one

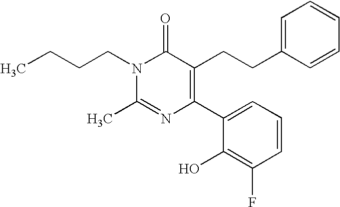

The title compound was prepared following the general procedures of Example 30 except substituting iodomethane with 1-iodobutane: ¹H NMR (400 MHz, CDCl₃) δ6.78-7.28 (m, 8H), 4.15 (t, 2H), 2.92-2.99 (m, 4H), 2.65 (s, 3H), 1.68-1.82 (m, 2H), 1.42-1.61 (m, 2H), 1.03 (t, 3H); LCMS (m/z): 381.2 (M+H).

### Example 33

### Preparation of 2-(dimethylamino)-6-(2-hydroxyphenyl)-5-(2-phenylethyl)-4(1H)-pyrimidinone

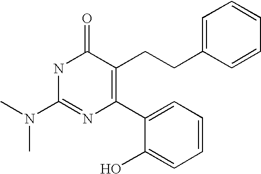

The title compound was prepared following the procedure of Example 1 except substituting acetamidine with 1,1-dimethyl guanidine sulfate and omission of the alkylation step (1c). LCMS (m/z): 336 (M+H).

### Example 34

### Preparation of 6-(2-hydroxyphenyl)-2-methyl-3-phenyl-5-(2-phenylethyl)-4(3H)-pyrimidinone

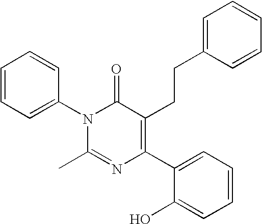

The title compound was prepared following the general procedure of Example 17 except substituting cyclopropylamine with aniline. LCMS (m/z): 383 (M+H).

### Example 35

### Preparation of 6-(3-fluoro-2-hydroxyphenyl)-3-heptyl-2-methyl-5-(2-phenylethyl)-4(3H)-pyrimidinone

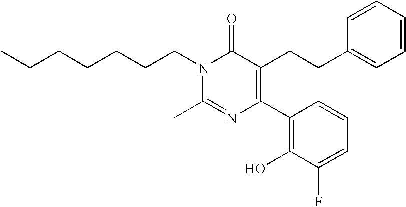

The title compound was prepared following the general procedures of Example 1 except substituting 1-bromoheptane for 1-bromopropane and substituting 3-fluoro-2-methoxybenzoic acid for 3-methoxybenzoic acid: ¹H NMR (400 MHz, CDCl₃) δ 10.2 (br s, 1H), 7.27 (m, 3H), 7.20 (m, 4H), 6.86 (m, 1H), 4.06 (m, 2H), 2.95 (s, 3H), 1.77 (m, 2H), 1.46 (m, 6H), 1.34 (m, 6H), 0.93 (m, 3H); LCMS (m/z): 423.4 (M+H).

### Example 36

### Preparation of 3-(1-benzothien-2-yl)-6-(3-fluoro-2-hydroxyphenyl)-2-methyl-5-(2-phenylethyl)-4(3H)-pyrimidinone

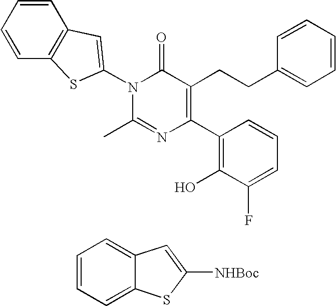

### a. 1,1-Dimethylethyl 1-benzothien-2-ylcarbamate

To a solution of 1-benzothiophene-2-carboxylic acid (5.0 g, 0.028 mol) in dry t-BuOH (70 mL) was added TEA (4.3 mL, 0.031 mol). After 5 min. of stirring, DPPA (6.67 mL, 0.031 mol) was added and the reaction was refluxed for 16 h. The reaction was concentrated and the resulting residue was diluted with ethyl acetate and washed successively with sat. NaHCO₃ and brine. The organic phase was dried over Na₂SO₄, filtered and concentrated before purifying by silica chromatography (0-40% ethyl acetate/hexane) to afford pure product (4.35 g) in 62% yield. ¹H NMR (400 MHz, DMSO-d₆) δ ppm 1.50 (s, 9H), 6.77 (s, 1H), 7.15 (t, J=0.85 Hz, 1H), 7.26 (t, J=0.85 Hz, 1H), 7.59 (d, J=7.79 Hz, 1H), 7.76 (d, J=7.78 Hz, 1H), 10.2 (brs, 1H).

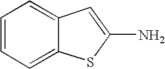

### b. 1-Benzothiophen-2-amine

To a solution of 1,1-Dimethylethyl 1-benzothien-2-ylcarbamate (1.0 g, 4.01 mmoles) in DCM (10 mL) was added TFA (2.0 mL) and stirred for 12 h. The reaction mixture was concentrated, and the resulted residue was redissolved in DCM and washed with 1N NaOH (2×50 mL), brine and dried over Na₂SO₄. The mixture was filtered and concentrated to afford pure product (0.54 g) in 91% yield. LCMS (m/z): 150.0 (M+H).

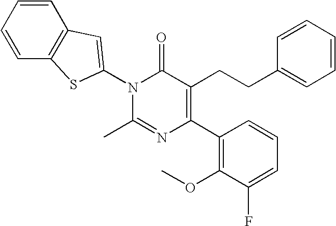

### c. 3-(1-Benzothien-2-yl)-6-[3-fluoro-2-(methyloxy)phenyl]-2-methyl-5-(2-phenylethyl)-4(3H)-pyrimidinone

The title compound was prepared according to the general procedure of Example 17 except substituting methyl 2-(methyloxy)benzoate with 3-fluoro-2-(methyloxy)benzoate and cyclopropanamine with 2-aminobenzothiophene. LCMS (m/z): 471.2 (M+H).

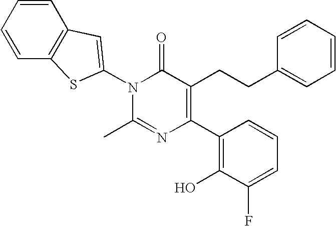

### d. 3-(1-Benzothien-2-yl)-6-(3-fluoro-2-hydroxyphenyl)-2-methyl-5-(2-phenylethyl)-4(3H)-pyrimidinone

A round bottom flask equipped with a stirring bar and a condenser was charged with 3-(-benzothien-2-yl)-6-[3-fluoro-2-(methyloxy)phenyl]-2-methyl-5-(2-phenylethyl)-4(3H)-pyrimidinone (3.33 g, 7.08 mmol). To this was added 80 mL of 45% HBr in acetic acid and 11 mL of water. The reaction was heated to 90° C. overnight. The crude residue was diluted with DCM and extracted with saturated sodium carbonate and brine. The organic layer was concentrated and purified by silica chromatography (0-30% ethyl acetate/hexane) to obtain the desired product (3.14 g) in 97% yield. LCMS (m/z): 457.2 (M+H).

### Example 37

### Preparation of 6-(3-fluoro-2-hydroxyphenyl)-2-methyl-3-(5-methyl-2-thienyl)-5-(2-phenylethyl)-4(3H)-pyrimidinone

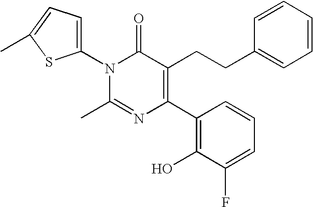

The title compound was prepared according to the general procedure of Example 17 except substituting methyl 2-(methyloxy)benzoate with 3-fluoro-2-(methyloxy)benzoate and cyclopropanamine with 2-amino-5-methylthiophene. LCMS (m/z): 421.0 (M+H).

### Example 38

### Preparation of 6-(3-fluoro-2-hydroxyphenyl)-2-methyl-3-(4-methyl-2-thienyl)-5-(2-phenylethyl)-4(3H)-pyrimidinone

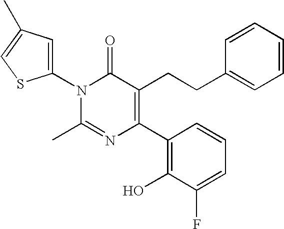

The title compound was prepared according to the general procedure of Example 17 except substituting methyl 2-(methyloxy)benzoate with 3-fluoro-2-(methyloxy)benzoate and cyclopropanamine with 2-amino-4-methylthiophene. LCMS (m/z): 421.2 (M+H).

### Example 39

### Preparation of 3-(4-biphenylyl)-6-(3-fluoro-2-hydroxyphenyl)-2-methyl-5-(2-phenylethyl)-4(3H)-pyrimidinone

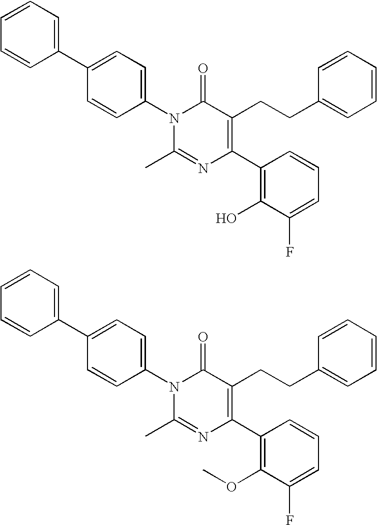

### a. 3-(4-Biphenylyl)-6-[3-fluoro-2-(methyloxy)phenyl]-2-methyl-5-(2-phenylethyl)-4(3H)-pyrimidinone

To a solution of 4-aminobiphenyl (0.34 g, 2.02 mmol) in toluene was added chlorotriisopropoxytitanium (0.48 mL, 3.03 mmol), and the mixture stirred for 30 min. A toluene solution of methyl (2Z)-3-(acetylamino)-3-[3-fluoro-2-(methyloxy)phenyl]-2-(2-phenylethyl)-2-propenoate (0.5 g, 1.35 mmoles) was added to the above mixture and the reaction was heated to reflux. Upon completion, the reaction was concentrated in vacuo, diluted with ethyl acetate, and washed sequentially with 1N HCl and brine. The organic layer was dried over Na₂SO₄, filtered and concentrated in vacuo. The residue was purified by silica chromatography (0-30% ethyl acetate/hexane) to afford the title compound (0.55 g, 83%). LCMS (m/z): 491.2 (M+H).

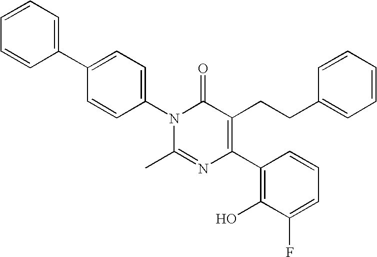

### b. 3-(4-Biphenylyl)-6-(3-fluoro-2-hydroxyphenyl)-2-methyl-5-(2-phenylethyl)-4(3H)-pyrimidinone

The title compound was prepared according to the general procedure described in Example 36d. LCMS (m/z): 477.2 (M+H).

### Example 40

### Preparation of 6-(3-fluoro-2-hydroxyphenyl)-2-methyl-5-(2-phenylethyl)-3-(5-phenyl-2-thienyl)-4(3H)-pyrimidinone

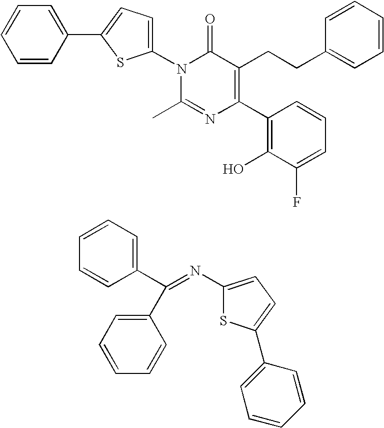

### a. N-(Diphenylmethylidene)-5-phenyl-2-thiophenamine

To a solution of 2-bromo-5-phenyl thiophene (3.00 g, 12.7 mmol) and 1,1 diphenylmethanimine (2.6 g, 15.2 mmol) in degassed toluene (55 mL) was added Pd₂(dba)₃ (1.16 g, 1.27 mmoles), and BINAP (2.37 g, 3.0 mmoles). The resulting solution was degassed again for 10 min. To this solution was added NaOtBu (1.71 g, 17.7 mmol), and the mixture was heated for 12 h at 80° C. The reaction mixture was concentrated in vacuum and purified by silica chromatography (30% ethyl acetate/hexanes) to afford 2.97 g of product (69%). LCMS (m/z): 340.2 (M+H).

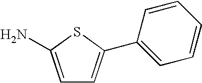

### b. 5-Phenyl-2-thiophenamine

To a solution of imine (2.97 g, 8.76 mmol) from Example 40a in THF (20 mL) was added 3N HCl (10 mL), and the mixture stirred at room temperature for 12 h. The reaction mixture was then concentrated in vacuum and triturated with ether. The resulting white solid was isolated by filtration and then dissolved in water. The aqueous pH was adjusted to 13 by the addition of 3N NaOH. The aqueous solution was extracted with DCM, washed with brine, dried (MgSO₄), filtered and concentrated to give 1.25 g of product in 81% yield. LCMS (m/z): 176.2 (M+H).

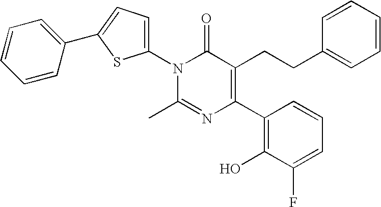

### c. 6-(3-Fluoro-2-hydroxyphenyl)-2-methyl-5-(2-phenylethyl)-3-(5-phenyl-2-thienyl)-4(3H)-pyrimidinone

The title compound was prepared according to the general procedure of Example 17 except substituting methyl 2-(methyloxy)benzoate with 3-fluoro-2-(methyloxy)benzoate and cyclopropanamine with 5-phenyl-2-thiophenamine. LCMS (m/z): 483.2 (M+H).

Examples Of The Biological Activity Of Reversed Pyrimidinones

The biological activity of the compounds of Formula (I) are demonstrated by the following tests:

(I) Calcium Receptor Inhibitor Assay

Calcilytic activity was measured by determining the IC₅₀ of test compounds for blocking increases of intracellular Ca²⁺ elicited by extracellular Ca²⁺ in HEK 293 4.0-7 cells stably expressing the human calcium receptor. HEK 293 4.0-7 cells were constructed as described by Rogers et al., J. Bone Miner. Res. 10 Suppl. 1:S483, 1995 (hereby incorporated by reference herein). Intracellular Ca²⁺ increases were elicited by increasing extracellular Ca²⁺ from 1 to 1.75 mM. Intracellular Ca²⁺ was measured using fluo-3, a fluorescent calcium indicator.

The procedure was as follows:

1. Cells were maintained in T-150 flasks in selection media (DMEM supplemented with 10% fetal bovine serum and 200 ug/mL hygromycin B), under 5% CO₂:95% air at 37° C. and were grown up to 90% confluency.

2. The medium was decanted and the cell monolayer was washed twice with phosphate-buffered saline (PBS) kept at 37° C. After the second wash, 6 mL of 0.02% EDTA in PBS was added and incubated for 4 minutes at 37° C. Following the incubation, cells were dispersed by gentle agitation.

3. Cells from 2 or 3 flasks were pooled and pelleted (100×g). The cellular pellet was resuspended in 10-15 mL of SPF−PCB+ and pelleted again by centrifugation. This washing was done twice.

Sulfate- and phosphate-free parathyroid cell buffer (SPF−PCB) contains 20 mM Na-Hepes, pH 7.4, 126 mM NaCl, 5 mM KCl, and 1 mM MgCl₂. SPF−PCB was made up and stored at 4° C. On the day of use, SPF−PCB was supplemented with 1 mg/mL of D-glucose and 1 mM CaCl₂ and then split into two fractions. To one fraction, bovine serum albumin (BSA; fraction V, ICN) was added at 5 mg/mL (SPF−PCB+). This buffer was used for washing, loading and maintaining the cells. The BSA-free fraction was used for diluting the cells in the cuvette for measurements of fluorescence.

4. The pellet was resuspended in 10 mL of SPF−PCB+ containing 2.2 uM fluo-3 (Molecular Probes) and incubated at room temperature for 35 minutes.

5. Following the incubation period, the cells were pelleted by centrifugation. The resulting pellet was washed with SPF−PCB+. After this washing, cells were resuspended in SPF−PCB+ at a density of 1-2×106 cells/mL.

6. For recording fluorescent signals, 300 uL of cell suspension were diluted in 1.2 mL of SPF buffer containing 1 mM CaCl₂ and 1 mg/mL of D-glucose. Measurements of fluorescence were performed at 37° C. with constant stirring using a spectrofluorimeter. Excitation and emission wavelengths were measured at 485 and 535 nm, respectively. To calibrate fluorescence signals, digitonin (5 mg/mL in ethanol) was added to obtain Fmax, and the apparent Fmin was determined by adding Tris-EGTA (2.5 M Tris-Base, 0.3 M EGTA). The concentration of intracellular calcium was calculated using the following equation:
Intracellular calcium=(F−Fₘᵢₙ/Fₘₐₓ)×K_{d}; where K_{d}=400 nM.

7. To determine the potential calcilytic activity of test compounds, cells were incubated with test compound (or vehicle as a control) for 90 seconds before increasing the concentration of extracellular Ca²⁺ from 1 to 2 mM. Calcilytic compounds were detected by their ability to block, in a concentration-dependent manner, increases in the concentration of intracellular Ca²⁺ elicited by extracellular Ca²⁺.

Compounds having an IC₅₀ value in the Calcium Receptor Inhibitor which are greater than 50 uM were considered to be inactive. Note that it is desirable for compounds to have lower IC₅₀ values in the Calcium Receptor Inhibitor Assay. For example, it is desirable for the compounds to have an IC₅₀ of 10 uM or lower, an IC₅₀ of 1 uM, and an IC₅₀ of 0.1 uM or lower.

(II) Calcium Receptor Binding Assay

HEK 293 4.0-7 cells stably transfected with the Human Parathyroid Calcium Receptor (“HuPCaR”) were scaled up in T180 tissue culture flasks. Plasma membrane is obtained by polytron homogenization or glass douncing in buffer (50 mM Tris-HCl pH 7.4, 1 mM EDTA, 3 mM MgCl₂) in the presence of a protease inhibitor cocktail containing 1 uM Leupeptin, 0.04 uM Pepstatin, and 1 mM PMSF. Aliquoted membrane was snap frozen and stored at −80° C. ³H labeled compound was radiolabeled to a radiospecific activity of 44 Ci/mmole and was aliquoted and stored in liquid nitrogen for radiochemical stability.

A typical reaction mixture contains 2 nM ³H compound ((R,R)—N-4′-Methoxy-t-3-3′-methyl-1′-ethylphenyl-1-(1-naphthyl)ethylamine), or ³H compound (R)—N-[2-Hydroxy-3-(3-chloro-2-cyanophenoxy)propyl]-1,1-dimethyl-2-(4-methoxyphenyl)ethylamine 4-10 ug membrane in homogenization buffer containing 0.1% gelatin and 10% EtOH in a reaction volume of 0.5 mL. Incubation is performed in 12×75 polyethylene tubes in an ice water bath. To each tube 25 uL of test sample in 100% EtOH is added, followed by 400 uL of cold incubation buffer, and 25 uL of 40 nM ³H-compound in 100% EtOH for a final concentration of 2 nM. The binding reaction is initiated by the addition of 50 uL of 80-200 ug/mL HEK 293 4.0-7 membrane diluted in incubation buffer, and allowed to incubate at 4° C. for 30 min. Wash buffer is 50 mM Tris-HCl containing 0.1% PEI. Nonspecific binding is determined by the addition of 100-fold excess of unlabeled homologous ligand, and is generally 20% of total binding. The binding reaction is terminated by rapid filtration onto 1% PEI pretreated GF/C filters using a Brandel Harvestor. Filters are placed in scintillation fluid and radioactivity assessed by liquid scintillation counting.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Without further elaboration, it is believed that one skilled in the art can use the preceding description to utilize the invention to its fullest extent. Therefore the Examples herein are to be construed as merely illustrative and not a limitation of the scope of the present invention in any way.

It will be apparent to those having skill in the art that changes may be made to the details of the above-described embodiments without departing from the underlying principles of the invention. Embodiments of the invention in which an exclusive property or privilege is claimed are defined as follows.

1. A compound according to formula (I) hereinbelow: 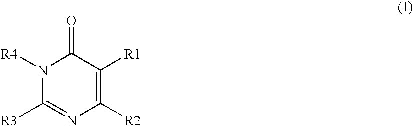
wherein:
R⁴ and R³ are independently one of: H, halogen, CN, CF₃, lower alkyl, cycloalky, or aryl; or R⁴ and R³ are together —(CH₂)ₙ— and n is 5, 4, or 3;
R² is an aryl group, which may have 0 to 4 substituents in the aryl ring and each substituent is at least one of: halogen, CN, CF₃, OCF₃, lower alkyl, N(lower alkyl)₂, lower alkoxy, OH, OC(O)-lower alkyl, OC(O)-lower alkylamino, or OC(O)-lower alkyl-N(lower alkyl)₂;
R¹ is one of H, lower alkyl, aryl or a group of the formula —(CH₂)ₙ—R⁵ wherein n is 0, 1, or 2; R⁵ is an aryl group which may have 0 to 3 substituents on the aryl ring and each substituent is at least one of: halogen, CN, CF₃, OCF₃, lower alkyl, lower alkoxy, NH-lower alkyl, NH-alkylaryl, N(lower alkyl)₂, OH, OC(O)-lower alk, OC(O)-lower alkylamino, or OC(O)-lower alkyl-N(lower alk)₂; or
pharmaceutically acceptable salts, hydrates, tautomers, solvates or complexes thereof.

2. The compound according to claim 1, wherein the compound is at least one of:
6-(2-hydroxyphenyl)-2-methyl-5-(2-phenylethyl)-3-propyl-4(3H)-pyrimidinone;
6-(2-hydroxyphenyl)-2-methyl-5-(2-phenylethyl)-3-ethyl-4(3H)-pyrimidinone;
6-(2-hydroxyphenyl)-2-methyl-5-(2-phenylethyl)-3-methyl-4(3H)-pyrimidinone;
6-(2-hydroxyphenyl)-2-methyl-5-(2-phenylethyl)-3-[2-(2-pyridinyl)ethyl]-4(3H)-pyrimidinone;
6-(2-hydroxyphenyl)-2-methyl-5-(2-phenylethyl)-3-butyl-4(3H)-pyrimidinone;
6-(2-hydroxyphenyl)-2-methyl-5-(2-phenylethyl)-3-pentyl-4(3H)-pyrimidinone;
6-(2-hydroxy-phenyl)-2-methyl-5-(2-phenethyl)-3-hexyl-3H-pyrimidin-4-one;
3-cyclopropylmethyl-6-(2-hydroxy-phenyl)-2-methyl-5-phenethyl-3H-pyrimidin-4-one;
3-(2-methylallyl)-6-(2-hydroxy-phenyl)-2-methyl-5-phenethyl-3H-pyrimidin-4-one;
3-(3-methylbutyl)-6-(2-hydroxy-phenyl)-2-methyl-5-phenethyl-3H-pyrimidin-4-one;
3-(2-cyclohexylethyl)-6-(2-hydroxy-phenyl)-2-methyl-5-phenethyl-3H-pyrimidin-4-one;
3-propyl-6-(3-flouro-2-hydroxy-phenyl)-2-methyl-5-phenethyl-3H-pyrimidin-4-one;
3-hexyl-6-(3-fluoro-2-hydroxy-phenyl)-2-methyl-5-phenethyl-3H-pyrimidin-4-one;
3-propyl-6-(2-hydroxy-phenyl)-2-methyl-5-(2-cyclohexylethyl)-3H-pyrimidin-4-one;
2-(2-hydroxyphenyl)-3-(2-phenylethyl)-6,7,8,9-tetrahydro-4H-pyrido[1,2-a]pyrimidin-4-one;
3-(2-cyclohexylethyl)-2-(2-hydroxyphenyl)-6,7,8,9-tetra hydro-4H-pyrido[1,2-a]pyrimidin-4-one;
3-cyclopropyl-6-(2-hydroxyphenyl)-2-methyl-5-(2-phenylethyl)pyrimidin-4(3H)-one;
6-(2-hydroxyphenyl)-2-methyl-3-[2-(1-methylpyrrolidin-2-yl)ethyl]-5-(2-phenylethyl)pyrimidin-4(3H)-one;
3-(2,2-dimethylpropyl)-6-(2-hydroxyphenyl)-2-methyl-5-(2-phenylethyl)pyrimidin-4(3H)-one;
3-sec-butyl-6-(2-hydroxyphenyl)-2-methyl-5-(2-phenylethyl)pyrimidin-4(3H)-one;
3-cyclopentyl-6-(2-hydroxyphenyl)-2-methyl-5-(2-phenylethyl)pyrimidin-4(3H)-one;
6-(2-hydroxyphenyl)-3-isobutyl-2-methyl-5-(2-phenylethyl)pyrimidin-4(3H)-one;
3-cyclobutyl-6-(2-hydroxyphenyl)-2-methyl-5-(2-phenylethyl)pyrimidin-4(3H)-one;
3-cyclohexyl-6-(2-hydroxyphenyl)-2-methyl-5-(2-phenylethyl)pyrimidin-4(3H)-one;
6-(2-hydroxyphenyl)-3-isopropyl-2-methyl-5-(2-phenylethyl)pyrimidin-4(3H)-one;
6-(2-hydroxyphenyl)-2-methyl-5-(2-phenylethyl)-3-(2,2,2-trifluoroethyl)pyrimidin-4(3H)-one;
6-(2-hydroxyphenyl)-2-methyl-3-octyl-5-(2-phenylethyl)pyrimidin-4(3H)-one;
3-heptyl-6-(2-hydroxyphenyl)-2-methyl-5-(2-phenylethyl)pyrimidin-4(3H)-one;
3-allyl-6-(2-hydroxyphenyl)-2-methyl-5-(2-phenylethyl)pyrimidin-4(3H)-one;
6-(3-fluoro-2-hydroxyphenyl)-2,3-dimethyl-5-(2-phenylethyl)pyrimidin-4(3H)-one;
3-ethyl-6-(3-fluoro-2-hydroxyphenyl)-2-methyl-5-(2-phenylethyl)pyrimidin-4(3H)-one;
3-butyl-6-(3-fluoro-2-hydroxyphenyl)-2-methyl-5-(2-phenylethyl)pyrimidin-4(3H)-one;
2-(dimethylamino)-6-(2-hydroxyphenyl)-5-(2-phenylethyl)-4(1H)-pyrimidinone;
6-(2-hydroxyphenyl)-2-methyl-3-phenyl-5-(2-phenylethyl)-4(3H)-pyrimidinone;
6-(3-fluoro-2-hydroxyphenyl)-3-heptyl-2-methyl-5-(2-phenylethyl)-4(3H)-pyrimidinone;
3-(1-benzothien-2-yl)-6-(3-fluoro-2-hydroxyphenyl)-2-methyl-5-(2-phenylethyl)-4(3H)-pyrimidinone;
6-(3-fluoro-2-hydroxyphenyl)-2-methyl-3-(5-methyl-2-thienyl)-5-(2-phenylethyl)-4(3H)-pyrimidinone;
6-(3-fluoro-2-hydroxyphenyl)-2-methyl-3-(4-methyl-2-thienyl)-5-(2-phenylethyl)-4(3H)-pyrimidinone;
3-(4-biphenylyl)-6-(3-fluoro-2-hydroxyphenyl)-2-methyl-5-(2-phenylethyl)-4(3H)-pyrimidinone; or
6-(3-fluoro-2-hydroxyphenyl)-2-methyl-5-(2-phenylethyl)-3-(5-phenyl-2-thienyl)-4(3H)-pyrimidinone.

3. A method of antagonizing a calcium receptor, which comprises administering to a subject in need thereof, an effective amount of a compound according to claim 1.

4. A method of treating a disease or disorder characterized by an abnormal bone or mineral homeostasis which comprises administering to a subject in need of treatment thereof an effective amount of a compound according to claim 1.

5. A method according to claim 1, wherein the bone or mineral disease or disorder is at least one of osteosarcoma, periodontal disease, fracture healing, osteoarthritis, joint replacement, rheumatoid arthritis, Paget's disease, humoral hypercalcemia, malignancy, or osteoporosis.

6. A method according to claim 1, wherein the bone or mineral disease or disorder is osteoporosis.

7. A method according to claim 6, wherein the compound is co-administered with an anti-resorptive agent.

8. A method according to claim 7, wherein the anti-resorptive agent is at least one of: estrogen, 1, 25 (OH)₂ vitamin D3, calcitonin, selective estrogen receptor modulators, vitronectin receptor antagonists, V−H+-ATPase inhibitors, src SH2 antagonists, bisphosphonates or cathepsin K inhibitors.

9. A method of increasing serum parathyroid levels which comprises administering to a subject in need of treatment an effective amount of a compound of claim 1.

10. A method according to claim 9, wherein the compound is co-administered with an anti-resorptive agent.

11. A method according to claim 10, wherein the anti-resorptive agent is at least one of: estrogen, 1, 25 (OH)₂ vitamin D3, calcitonin, selective estrogen receptor modulators, vitronectin receptor antagonists, V−H+-ATPase inhibitors, src SH2 antagonists, bisphosphonates or cathepsin K inhibitors.

12. A pharmaceutical composition comprising a compound according to claim 1.
